(19) United States
(12) Patent Application Publication
(10) Pub. No.: US 2026/0083714 A1
(43) Pub. Date: Mar. 26, 2026

(54) DOSING REGIMEN FOR TREATING PKU WITH A PIPERIDINE INHIBITOR OF SLC6A19 FUNCTION

(71) Applicant: Jnana Therapeutics Inc., Boston, MA (US)

(72) Inventors: John Peter Throup, New Hope, PA (US); Heather Smith Blanchette, Milledgeville, GA (US)

(21) Appl. No.: 19/111,597

(22) PCT Filed: Aug. 25, 2023
(86) PCT No.: PCT/US23/31169

§ 371 Date: Mar. 13, 2025

Related U.S. Application Data
(60) Provisional application No. 63/406,446, filed on Sep. 14, 2022.

(51) Int. Cl. A61K 31/445

(52) U.S. Cl. CPC A61K 31/445

(57) ABSTRACT

Disclosed are methods of treating phenylketonuria, comprising administering to a patient in need thereof an effective amount of the following compound:

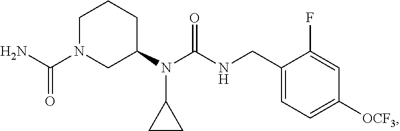 [SMILES: NC(=O)N1CCC[C@@H](N(C(=O)NCc2ccc(OC(F)(F)F)cc2F)C2CC2)C1]

or a pharmaceutically acceptable salt thereof

### RELATED APPLICATION

This application claims the benefit of priority to U.S. Provisional Patent Application No. 63/406,446, filed Sep. 14, 2022.

### BACKGROUND

Phenylketonuria (PKU) is an inborn error of metabolism caused by mutations in phenylalanine hydroxylase (PAH), the enzyme responsible for metabolizing phenylalanine. PKU is an autosomal recessive metabolic disorder in which phenylalanine is not properly metabolized and results in abnormally high levels of plasma phenylalanine. People who have PKU have abnormally high blood levels of phenylalanine, which if untreated can lead to irreversible neurological damage resulting in a spectrum of complications such as intellectual disabilities, seizures, neurodevelopmental and behavioral disorders. PKU is difficult to treat because blood levels of phenylalanine are directly related to diet. Patients must adhere to a life-long and strict diet that impacts all aspects of patients' lives. Current standard of care are enzyme co-factor and enzyme substitution therapy but these therapies are not effective in all patients, and carry potential risk for adverse events.

The enzyme responsible for metabolizing phenylalanine, and thus maintaining phenylalanine homeostasis is phenylalanine hydroxylase (PAH). Loss-of-function (LOF) mutations at PAH gene at chromosome 12q23.2 are known to cause most forms of PKU. These LOF mutations resulting in PKU can be diagnosed as classical PKU (the most severe form), and “mild PKU” or “hyperphe” a less severe form. In addition to PAH, mutations in other enyzmes that affect phenylalanine metabolism, such as dihydropteridine reductase (DHPR), the enzyme responsible for synthesis of co-factors required for PAH activity, may also result in elevated levels of phenylalanine. In addition to diet, blood amino acid levels, including levels of phenylalanine, are regulated by SLC6A19. SCL6A19 is located in the proximal tubule of the kidney and is responsible for reabsorption of amino acids back into the blood.

### SUMMARY

One aspect of the invention provides a method and dosing regimen useful for treating or preventing a disease or disorder associated with abnormal levels of amino acids by modulation of SLC6A19 transport.

Another aspect of the invention provides a method and dosing regimen useful for treating or preventing a disease or disorder associated with a genetic defect in phenylalanine hydroxylase.

Accordingly, provided herein is a method of treating or preventing a disease or disorder associated with a genetic defect in phenylalanine hydroxylase, comprising orally administering to a patient in need thereof an effective amount of the following compound, or a pharmaceutically acceptable salt thereof:

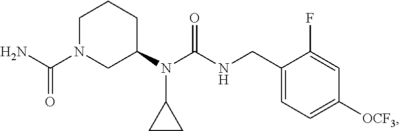 [SMILES: NC(=O)N1CCC[C@@H](N(C(=O)NCc2ccc(OC(F)(F)F)cc2F)C2CC2)C1]

wherein the compound is formulated as a tablet.

Also provided herein is a method of treating or preventing a disease or disorder associated with a genetic defect in phenylalanine hydroxylase, comprising orally administering to a patient in need thereof an effective amount of the following compound, or a pharmaceutically acceptable salt thereof:

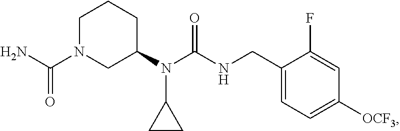 [SMILES: NC(=O)N1CCC[C@@H](N(C(=O)NCc2ccc(OC(F)(F)F)cc2F)C2CC2)C1]

wherein the compound is formulated as a suspension.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features, objects, and advantages of the invention will be apparent from the detailed description, and from the claims.

### BRIEF DESCRIPTION OF THE DRAWINGS

FIG. 1 is a graph of the pharmacodynamic effect of Compound 1 in healthy volunteers showing changes in total phenylalanine or overall SLC6A19 amino acid substrates (Hartnup amino acids) excretion over 24 hours following administration of a single dose of the compound (10-170 mg) in the form of a suspension.

FIG. 2 is an assessment of the pharmacokinetics of Compound 1 in healthy volunteers over 24 hours after 14 days of administration of a suspension of the compound at three different doses (25 or 75 mg twice daily [BID] or 150 mg once daily [QD]).

FIG. 3A is a graph of the pharmacodynamic effect of Compound 1 over time in healthy volunteers by measuring changes in total overall SLC6A19 amino acid substrates (Hartnup amino acids) over 14 days of administration of a suspension of the compound at three different doses (25 or 75 mg twice daily [BID] or 150 mg once daily [QD]).

FIG. 3B is a graph of the pharmacodynamic effect of Compound 1 over time in healthy volunteers by measuring changes in total 24 hour phenylalanine over 14 days of administration of a suspension of the compound at three different doses (25 or 75 mg twice daily [BID] or 150 mg once daily [QD]).

FIG. 4 is a graph of the pharmacokinetics of Compound 1 over 72 hours in healthy volunteers in fed or fasted states when administering 100 mg of the compound as a suspension formulation or a tablet.

### DETAILED DESCRIPTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

Additional asymmetric carbon atoms may be present in a substituent such as an alkyl group. All such isomers, as well as mixtures thereof, are intended to be included in this invention.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention.

For purposes of this invention, the chemical elements are identified in accordance with the Periodic Table of the Elements, CAS version, Handbook of Chemistry and Physics, 67th Ed., 1986-87, inside cover.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, a therapeutic that “prevents” or “reduces the risk of developing” a disease, disorder, or condition refers to a compound that, in a statistical sample, reduces the occurrence of the disease, disorder, or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, the term “modulate” includes up-regulation and down-regulation, e.g., enhancing or inhibiting a response.

### Compound of the Invention

In certain embodiments, the compound used in the methods of the present invention is Compound 1 i.e. (R)-3-(1-cyclopropyl-3-(2-fluoro-4-(trifluoromethoxy)benzyl)ureido) piperidine-1-carboxamide, which has the structure:

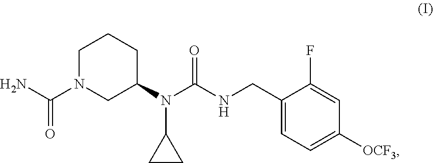 [SMILES: NC(=O)N1CCC[C@@H](N(C(=O)NCc2ccc(OC(F)(F)F)cc2F)C2CC2)C1]

or a pharmaceutically acceptable salt thereof. The compound is a SLC6A19 inhibitor. For example, the compound reduces systemic phenylalanine levels in the subject.

### Pharmaceutical Compositions

In certain embodiments, the invention relates to a pharmaceutical composition, comprising the compound of the invention and a pharmaceutically acceptable carrier for treating or preventing a disease or disorder associated with a genetic defect in phenylalanine hydroxylase.

In certain embodiments, the pharmaceutical composition is formulated as a tablet. In other embodiment, the pharmaceutical composition is formulated as a suspension.

The compositions and methods of the present invention may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, suspension, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (al-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (μm), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example, as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt or cocrystal. When used in medicine the salts or cocrystals should be pharmaceutically acceptable, but non-pharmaceutically acceptable salts or cocrystals may conveniently be used to prepare pharmaceutically acceptable salts or cocrystals thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### Methods of Treatment

One aspect of the invention provides a method and dosing regimen useful for treating or preventing a disease or disorder associated with abnormal levels of amino acids by modulation of SLC6A19 transport.

Another aspect of the invention provides a method and dosing regimen useful for treating or preventing a disease or disorder associated with a genetic defect in phenylalanine hydroxylase.

One aspect of the invention provides a method of treating or preventing a disease or disorder associated with a genetic defect in phenylalanine hydroxylase, comprising orally administering to a patient in need thereof an effective amount of the following compound, or a pharmaceutically acceptable salt thereof:

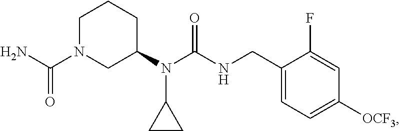 [SMILES: NC(=O)N1CCC[C@@H](N(C(=O)NCc2ccc(OC(F)(F)F)cc2F)C2CC2)C1]

wherein the compound is formulated as a tablet.

In certain embodiments, the amount of compound is about 5 mg/day to about 1025 mg/day.

In certain embodiments, the amount of compound is about 75 mg/day to about 1025 mg/day.

In certain embodiments, the amount of the compound is about 100 mg/day to about 500 mg/day. In other embodiments, the amount of the compound is about 500 mg/day to about 1000 mg/day. In other embodiments, the amount of the compound is about 100 mg/day to about 200 mg/day. In other embodiments, the amount of the compound is about 200 mg/day to about 300 mg/day. In other embodiments, the amount of the compound is about 300 mg/day to about 400 mg/day. In other embodiments, the amount of the compound is about 400 mg/day to about 500 mg/day. In other embodiments, the amount of the compound is about 500 mg/day to about 600 mg/day. In other embodiments, the amount of the compound is about 600 mg/day to about 700 mg/day. In other embodiments, the amount of the compound is about 700 mg/day to about 800 mg/day. In other embodiments, the amount of the compound is about 800 mg/day to about 900 mg/day. In other embodiments, the amount of the compound is about 900 mg/day to about 1000 mg/day.

In certain embodiments, the amount of the compound is about 100 mg/day, about 125 mg/day, about 150 mg/day, or about 175 mg/day. In other embodiments, the amount of the compound is about 200 mg/day, about 225 mg/day, about 250 mg/day, or about 275 mg/day. In other embodiments, the amount of the compound is about 300 mg/day, about 325 mg/day, about 350 mg/day, or about 375 mg/day. In other embodiments, the amount of the compound is about 400 mg/day, about 425 mg/day, about 450 mg/day, about 475 mg/day, or about 500 mg/day. In other embodiments, the amount of the compound is about 500 mg/day, about 525 mg/day, about 550 mg/day, or about 575 mg/day. In other embodiments, the amount of the compound is about 600 mg/day, about 625 mg/day, about 650 mg/day, or about 675 mg/day. In other embodiments, the amount of the compound is about 700 mg/day, about 725 mg/day, about 750 mg/day, or about 775 mg/day. In other embodiments, the amount of the compound is about 800 mg/day, about 825 mg/day, about 850 mg/day, or about 875 mg/day. In other embodiments, the amount of the compound is about 900 mg/day, about 925 mg/day, about 950 mg/day, about 975 mg/day, or about 1000 mg/day.

In certain embodiments, the amount of compound is about 5 mg/day to about 100 mg/day.

In certain embodiments, the amount of the compound is about 5 mg/day to about 25 mg/day. In other embodiments, the amount of the compound is about 25 mg/day to about 50 mg/day. In other embodiments, the amount of the compound is about 50 mg/day to about 75 mg/day.

In certain embodiments, the amount of the compound is about 5 mg/day, about 10 mg/day, about 25 mg/day, about 50 mg/day, or about 75 mg/day. In certain embodiments, the % w/w of the compound in the tablet is about 5% to about 30% w/w.

In certain embodiments, the % w/w of the compound in the tablet is about 5% to about 10% w/w. In other embodiments, the % w/w of the compound in the tablet is about 20% to about 30% w/w.

In certain embodiments, each component of the tablet is intragranular.

In certain embodiments, at least one component of the tablet, other than the compound, is extragranular.

In certain embodiments, the tablet is a coated tablet.

In certain embodiments, the amount of the compound is administered in one dose per day. In other embodiments, the amount of the compound is administered in two equally divided doses per day.

In certain embodiments, the compound is administered once per day (QD). In other embodiments, the amount of the compound is administered twice per day (BID).

In certain embodiments, 25 mg of the compound is administered twice per day (BID).

In certain embodiments, 75 mg of the compound is administered twice per day (BID).

In certain embodiments, 150 mg of the compound is administered once per day (QD).

Another aspect of the invention provides a method of treating or preventing a disease or disorder associated with a genetic defect in phenylalanine hydroxylase, comprising orally administering to a patient in need thereof an effective amount of the following compound, or a pharmaceutically acceptable salt thereof:

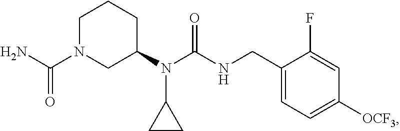 [SMILES: NC(=O)N1CCC[C@@H](N(C(=O)NCc2ccc(OC(F)(F)F)cc2F)C2CC2)C1]

wherein the compound is formulated as a suspension.

In certain embodiments, the suspension is an oral suspension.

In certain embodiments, the suspension is formulated for oral administration.

In certain embodiments, the amount of compound is about 5 mg/day to about 1525 mg/day.

In certain embodiments, the amount of compound is about 10 mg/day to about 750 mg/day.

In certain embodiments, the amount of the compound is about 10 mg/day to about 525 mg/day.

In certain embodiments, the amount of the compound is about 25 mg/day to about 100 mg/day. In other embodiments, the amount of the compound is about 150 mg/day to about 250 mg/day. In other embodiments, the amount of the compound is about 300 mg/day to about 500 mg/day.

In certain embodiments, the amount of the compound is about 25 mg/day. In other embodiments, the amount of the compound is about 75 mg/day. In other embodiments, the amount of the compound is about 150 mg/day. In other embodiments, the amount of the compound is about 250 mg/day. In other embodiments, the amount of the compound is about 300 mg/day. In other embodiments, the amount of the compound is about 500 mg/day. In other embodiments, the amount of compound is about 750 mg/day to about 1000 mg/day.

In certain embodiments, the amount of the compound is about 1000 mg/day to about 1500 mg/day. In other embodiments, the amount of the compound is about 1000 mg/day. In other embodiments, the compound is about 1500 mg/day.

In certain embodiments, the % w/w of the compound in the suspension is about 25% to about 95% w/w.

In certain embodiments, the % w/w of the compound in the t suspension is about 33% to about 60% w/w. In other embodiments, the % w/w of the compound in the suspension is about 75% to about 90% w/w. In other embodiments, the % w/w of the compound in the suspension is about 90% to about 95% w/w.

In certain embodiments, the suspension consists of the compound, a wetting agent, and a vehicle.

In certain embodiments, the amount of the compound is administered in one dose per day. In other embodiments, the amount of the compound is administered in two equally divided doses per day.

In certain embodiments, the compound is administered once per day (QD). In other embodiments, the amount of the compound is administered twice per day (BID).

In certain embodiments, 25 mg of the compound is administered twice per day (BID).

In certain embodiments, 75 mg of the compound is administered twice per day (BID).

In certain embodiments, 150 mg of the compound is administered once per day (QD).

In certain embodiments, the treatment continues indefinitely (i.e., chronic treatment).

In certain embodiments, the patient is a human.

In certain embodiments, the patient is an adult. In other embodiments, the patient is an elderly adult. In other embodiments, the patient is a child. In other embodiments, the patient is an infant or a toddler. In other embodiments, the patient is a male. In other embodiments, the patient is a female.

In certain embodiments, the human is of African ancestry, Asian ancestry, Caucasian ancestry, American Indian ancestry, Hispanic/Latino ancestry, or Pacific Islander ancestry.

In certain embodiments, the concentration of phenylalanine in the blood of the patient is reduced relative to pretreatment baseline.

In certain embodiments, the concentration of phenylalanine is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In certain embodiments, the concentration of phenylalanine is reduced by at least 10%, at least 20%, at least 30%, at least 40%, at least 50%, at least 60%, at least 70%, at least 80%, or at least 90%.

In certain embodiments, the disease or disorder associated with a genetic defect in phenylalanine hydroxylase is phenylketonuria.

In certain embodiments, the phenylketonuria is classical phenylketonuria. In other embodiments, the phenylketonuria is mild phenylketonuria.

In certain embodiments, the disease or disorder associated with a genetic defect in phenylalanine hydroxylase is hyperphenylalaninemia.

In certain embodiments, the compound inhibits SLC6A19 in the patient.

### EXAMPLES

The invention is further described in the following examples, which do not limit the scope of the invention described in the claims.

### Example 1. Oral Suspension Formulations of the Compound

### Preparation of Oral Suspensions

The various concentrations of Compound 1 oral suspensions will be prepared for each dose level. Each suspension preparation will be made for the individual volunteer to administer a specific dose. All oral suspensions are prepared by adding the diluent, OraBlend® SF (manufactured by Padagis LLC (formerly Perrigo® Company plc) containing 0.1% w/v sodium lauryl sulfate. The compositions of the various oral suspensions to provide each dose level: 25 mg, 75 mg, 150 mg, 250 mg, 350 mg and 500 mg, to be prepared at the compounding pharmacy are presented in Table 1 to Table 7, respectively.

TABLE 1
Composition of Compound 1 Oral Suspension, 25 mg

| Component | Function | Composition | |
|---|---|---|---|
| Compound 1 | Active | 25 | mg |
| Sodium Lauryl Sulfate | Wetting agent | 25 | mg |
| OraBlend ® SF | Vehicle | 25 | mL |

TABLE 2
Composition of Compound 1 Oral Suspension, 25 mg

| Component | Function | Composition | |
|---|---|---|---|
| Compound 1 | Active | 75 | mg |
| Sodium Lauryl Sulfate | Wetting agent | 25 | mg |
| OraBlend ® SF | Vehicle | 25 | mL |

TABLE 3
Composition of Compound 1 Oral Suspension, 150 mg

| Component | Function | Composition | |
|---|---|---|---|
| Compound 1 | Active | 150 | mg |
| Sodium Lauryl Sulfate | Wetting agent | 25 | mg |
| OraBlend ® SF | Vehicle | 25 | mL |

TABLE 4
Composition of Compound 1 Oral Suspension, 250 mg

| Component | Function | Composition | |
|---|---|---|---|
| Compound 1 | Active | 250 | mg |
| Sodium Lauryl Sulfate | Wetting agent | 25 | mg |
| OraBlend ® SF | Vehicle | 25 | mL |

TABLE 5
Composition of Compound 1 Oral Suspension, 300 mg

| Component | Function | Composition | |
|---|---|---|---|
| Compound 1 | Active | 300 | mg |
| Sodium Lauryl Sulfate | Wetting agent | 25 | mg |
| OraBlend ® SF | Vehicle | 25 | mL |

TABLE 6
Composition of Compound 1 Oral Suspension, 300 mg

| Component | Function | Composition | |
|---|---|---|---|
| Compound 1 | Active | 500 | mg |
| Sodium Lauryl Sulfate | Wetting agent | 25 | mg |
| OraBlend ® SF | Vehicle | 25 | mL |

TABLE 7
Composition of Compound 1 Oral Suspension,
for any mg dose between 10 mg and 750 mg

| Component | Function | Composition | |
|---|---|---|---|
| Compound 1 | Active | 10-750 | mg |
| Sodium Lauryl Sulfate | Wetting agent | 25 | mg |
| OraBlend ® SF | Vehicle | 25 | mL |

The following procedure is used to prepare the oral suspensions at the compounding pharmacy.
1. Weigh the required quantity of Compound 1 drug substance on a tared weighing paper/boat.
2. Transfer the weighed Compound 1 to a 50 mL Falcon or equivalent tube.
3. Prepare the vehicle (OraBlend© SF with 0.1% sodium lauryl sulfate) by weighing appropriate amount of sodium lauryl sulfate and dissolving it in OraBlend© SF with mixing at room temperature.
4. Dispense 25 mL of OraBlend© SF with 0.1% sodium lauryl sulfate to the Falcon tube or equivalent containing the weighed Compound 1 drug substance and tightly cap the tube.
5. Gently vortex the contents of the tube for at least 30 seconds to mix and suspend the Compound 1 in the OraBlend® SF with 0.1% sodium lauryl sulfate.
6. Set tube with its contents upright.
7. Prior to administration of the dose to the subject gently hand agitate the contents of the tube to resuspend the Compound 1 particles.
8. Once the contents of the tube are administered, the tube will be washed with 2×25 mL Purified Water USP and the washings will be administered to the subject.

### Example 2. Tablet Formulations of the Compound

### Preparation of Tablets

Two tablet formulations comprising Compound 1 in an amount of 75 mg are described in Tables 8 and 9.

TABLE 8
Tablet formation A: 75 mg strength, 300 mg tablet

| | | | |
|---|---|---|---|
| 75 mg Strength | Grade | % w/w | mg/tab |

Intragranular

| | | | |
|---|---|---|---|
| Compound 1 | | 25.00 | 75.00 |
| Microcrystalline Cellulose | Avicel PH 102 | 34.00 | 102.00 |
| Mannitol | Pearlitol 100 SD | 34.00 | 102.00 |
| Croscarmellose Sodium | Ac-Di-Sol SD711 | 3.00 | 9.00 |
| Magnesium Stearate | Ligamed MF-2-K | 0.50 | 1.50 |
| Silicon Dioxide | Aerosil 200 Pharma | 0.25 | 0.75 |
| Sodium Lauryl Sulfate | Kolliphor SLS Fine | 0.50 | 1.50 |
| Magnesium Stearate | Ligamed MF-2-K | 0.50 | 1.50 |
| Croscarmellose Sodium | Ac-Di-Sol SD711 | 2.00 | 6.00 |
| Silicon Dioxide | Aerosil 200 Pharma | 0.25 | 0.75 |

| | | |
|---|---|---|
| Total | 100.00 | 300.00 |

TABLE 9
Tablet Formation B: 75 mg strength, 300 mg tablet

| 75 mg Strength | Grade | % w/w | mg/tab |
|---|---|---|---|
| Intragranular | | | |
| Compound 1 | | 25.00 | 75.00 |
| Microcrystalline Cellulose | Avicel PH 102 | 34.00 | 102.00 |
| Mannitol | Pearlitol 100 SD | 34.00 | 102.00 |
| Crospovidone | Polyplasdone Ultra 10 | 3.00 | 9.00 |
| Magnesium Stearate | Ligamed MF-2-K | 0.50 | 1.50 |
| Silicon Dioxide | Aerosil 200 Pharma | 0.25 | 0.75 |
| Sodium Lauryl Sulfate | Kolliphor SLS Fine | 0.50 | 1.50 |
| Magnesium Stearate | Ligamed MF-2-K | 0.50 | 1.50 |
| Crospovidone | Polyplasdone Ultra 10 | 2.00 | 6.00 |
| Silicon Dioxide | Aerosil 200 Pharma | 0.25 | 0.75 |

| | | |
|---|---|---|
| Total | 100.00 | 300.00 |

Two coated tablet formulations comprising Compound 1 in an amount of 75 mg are described in Tables 10 and 11.

TABLE 10
Tablet Formation C: 75 mg strength, 300 mg tablet (pre-coated)

| | | | |
|---|---|---|---|
| 75 mg Strength | Grade | % w/w | mg/tab |

Intragranular

| | | | |
|---|---|---|---|
| Compound 1 | | 25.0 | 75.0 |
| Microcrystalline Cellulose | Avicel PH 102 | 33.625 | 100.9 |
| Mannitol | Pearlitol 100 SD | 33.625 | 100.9 |
| Croscarmellose Sodium | Ac-Di-Sol SD711 | 3.0 | 9.0 |
| Magnesium Stearate | Ligamed MF-2-K | 0.75 | 2.3 |
| Silicon Dioxide | Aerosil 200 Pharma | 0.75 | 2.3 |
| Sodium Lauryl Sulfate | Kolliphor SLS Fine | 0.5 | 1.5 |

Extragranular

| | | | |
|---|---|---|---|
| Magnesium Stearate | Ligamed MF-2-K | 0.5 | 1.5 |
| Croscarmellose Sodium | Ac-Di-Sol SD711 | 2.0 | 6.0 |
| Silicon Dioxide | Aerosil 200 Pharma | 0.25 | 0.8 |

| | | |
|---|---|---|
| Total (Core tablet) | 100.0 | 300.0 |

Coating

| | | | |
|---|---|---|---|
| Opadry QX Film Coat | Opadry QX White | 3.0-4.0 | 9.0-12.0 |
| | 321A180025 | | |

| | |
|---|---|
| Total (Coated tablet) | 309.0-312.0 |

TABLE 11
Tablet Formation C: 25 mg strength, 300 mg tablet (pre-coated)

| | | | |
|---|---|---|---|
| 25 mg Strength | Grade | % w/w | mg/tab |

Intragranular

| | | | |
|---|---|---|---|
| Compound 1 | | 8.33 | 25.0 |
| Microcrystalline Cellulose | Avicel PH 102 | 41.96 | 125.875 |
| Mannitol | Pearlitol 100 SD | 41.96 | 125.875 |
| Croscarmellose Sodium | Ac-Di-Sol SD711 | 3.0 | 9.0 |
| Magnesium Stearate | Ligamed MF-2-K | 0.75 | 2.25 |
| Silicon Dioxide | Aerosil 200 Pharma | 0.75 | 2.25 |
| Sodium Lauryl Sulfate | Kolliphor SLS Fine | 0.5 | 1.5 |

Extragranular

| | | | |
|---|---|---|---|
| Magnesium Stearate | Ligamed MF-2-K | 0.5 | 1.5 |
| Croscarmellose Sodium | Ac-Di-Sol SD711 | 2.0 | 6.0 |
| Silicon Dioxide | Aerosil 200 Pharma | 0.25 | 0.75 |

| | | |
|---|---|---|
| Total (Core tablet) | 100.0 | 300.0 |

Coating

| | | | |
|---|---|---|---|
| Opadry QX Film Coat | Opadry QX White | 3.0-4.0 | 9.0-12.0 |
| | 321A180025 | | |

| | |
|---|---|
| Total (Coated tablet) | 309.0-312.0 |

Patients will be administered a combination of 75 mg and 25 mg tablets in order to achieve the following final doses (Table 12). The tablets will be administered once or twice daily.

TABLE 12
Combinations of Tablets for Final Doses

| | Quantity of 75 mg | Quantity of 25 mg |
|---|---|---|
| | (Compound 1) tablet | (Compound 1) tablet |
| Final Dose | administered | administered |
| 100 | 1 | 1 |
| 125 | 1 | 2 |
| 150 | 2 | 0 |
| 175 | 2 | 1 |
| 200 | 2 | 2 |
| 225 | 3 | 0 |
| 250 | 3 | 1 |
| 275 | 3 | 2 |
| 300 | 4 | 0 |
| 325 | 4 | 1 |
| 350 | 4 | 2 |
| 375 | 5 | 0 |
| 400 | 5 | 1 |
| 425 | 5 | 2 |
| 450 | 6 | 0 |
| 475 | 6 | 1 |
| 500 | 6 | 2 |

### Example 3. Administration of Suspension Formulation

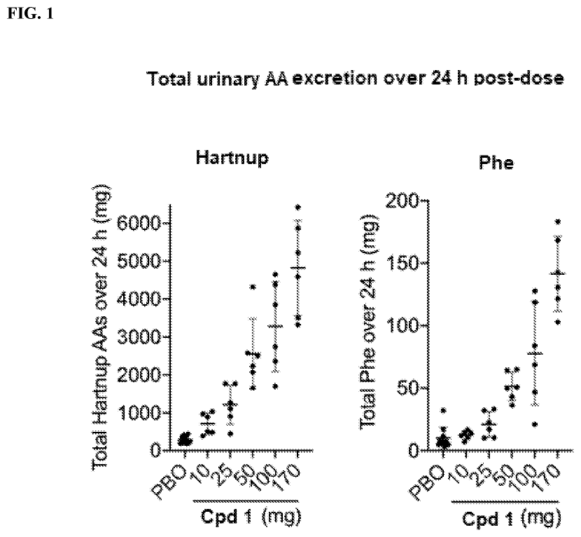

A suspension formulation of varying doses of Compound 1 (10-170 mg) was administered to healthy volunteers. Changes in amino acid excretion over 24 h following a single dose were measured (FIG. 1). Up to a 14-fold increase in total Phe excretion (from 10 mg to 142 mg) and a 16-fold increase in Hartnup AA excretion (from 0.29 g to 4.8 g) were observed over 24 hr after dosing (Tables 13A and 13B).

TABLE 13A
Excretion amounts (mg) over 24 h (mg)
Hartnup Amino Acids

| Treatment (Dose) | Mean | SD |
|---|---|---|
| Placebo | 291.75 | 92.82 |
| Cpd 1 (10 mg) | 712.45 | 282.78 |
| Cpd 1 (25 mg) | 1213.02 | 512.21 |
| Cpd 1 (50 mg) | 2560.22 | 922.84 |
| Cpd 1 (100 mg) | 3281.75 | 1186.92 |
| Cpd 1 (170 mg) | 4823.71 | 1252.52 |

TABLE 13B
Excretion amounts (mg) over 24 h Phenylalanine

| Treatment (Dose) | Mean | SD |
|---|---|---|
| Placebo | 10.22 | 8.70 |
| Cpd 1 (10 mg) | 12.50 | 3.41 |
| Cpd 1 (25 mg) | 21.02 | 10.24 |
| Cpd 1 (50 mg) | 51.53 | 11.37 |
| Cpd 1 (100 mg) | 77.84 | 41.14 |
| Cpd 1 (170 mg) | 141.61 | 29.88 |

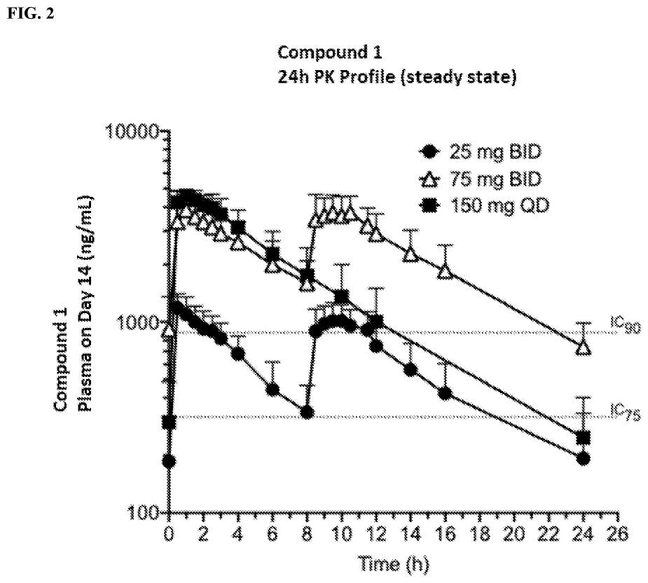

A suspension formulation of varying doses of Compound 1 (25 or 75 mg twice daily [BID] or 150 mg once daily [QD]) was administered to healthy volunteers. Plasma levels of Compound 1 over 24 hours after 14 days of administration were measured (FIG. 2). Post-dose plasma levels are recited in Table 14.

TABLE 14
Plasma

| Treatment (Dose) | Time Post-Dose (h) | Mean (ng/mL) | SD |
|---|---|---|---|
| Cpd 1 25 mg (BID) | 0 | 186.06 | 131.19 |
| | 0.5 | 1185.10 | 218.43 |
| | 1 | 1094.74 | 256.38 |
| | 1.5 | 999.62 | 207.35 |
| | 2 | 921.94 | 219.63 |
| | 2.5 | 898.51 | 174.87 |
| | 3 | 820.37 | 164.49 |
| | 4 | 682.14 | 162.44 |
| | 6 | 441.90 | 175.12 |
| | 8 | 336.39 | 129.24 |
| | 8.5 | 894.15 | 275.37 |
| | 9 | 977.28 | 234.61 |
| | 9.5 | 1011.54 | 247.06 |
| | 10 | 1012.83 | 218.12 |
| | 10.5 | 957.03 | 203.51 |
| | 11.5 | 909.02 | 225.62 |
| | 12 | 748.10 | 216.91 |
| | 14 | 561.84 | 209.83 |
| | 16 | 422.44 | 184.48 |
| | 24 | 192.84 | 140.37 |
| Cpd 1 75 mg (BID) | 0 | 915.59 | 441.51 |
| | 0.5 | 3348.74 | 1547.83 |
| | 1 | 3861.91 | 976.48 |
| | 1.5 | 3549.36 | 929.27 |
| | 2 | 3334.10 | 891.12 |
| | 2.5 | 3139.84 | 805.20 |
| | 3 | 2911.22 | 662.85 |
| | 4 | 2611.99 | 714.55 |
| | 6 | 1995.13 | 656.95 |
| | 8 | 1599.37 | 493.51 |
| | 8.5 | 3432.96 | 1214.93 |
| | 9 | 3604.79 | 850.86 |
| | 9.5 | 3740.26 | 880.62 |
| | 10 | 3595.84 | 935.21 |
| | 10.5 | 3735.29 | 818.17 |
| | 11.5 | 3196.40 | 761.31 |
| | 12 | 2899.17 | 787.54 |
| | 14 | 2283.86 | 739.47 |
| | 16 | 1852.51 | 667.68 |
| | 24 | 740.56 | 245.63 |
| Cpd 1 150 mg (QD) | 0 | 298.51 | 187.36 |
| | 0.5 | 4233.40 | 594.09 |
| | 1 | 4587.92 | 337.96 |
| | 1.5 | 4362.07 | 520.63 |
| | 2 | 4104.60 | 491.97 |
| | 2.5 | 3936.35 | 735.67 |
| | 3 | 3660.01 | 764.86 |
| | 4 | 3129.73 | 724.08 |
| | 6 | 2273.28 | 707.00 |
| | 8 | 1747.77 | 697.60 |
| | 10 | 1358.84 | 641.54 |
| | 12 | 1003.51 | 500.61 |
| | 24 | 247.39 | 154.66 |

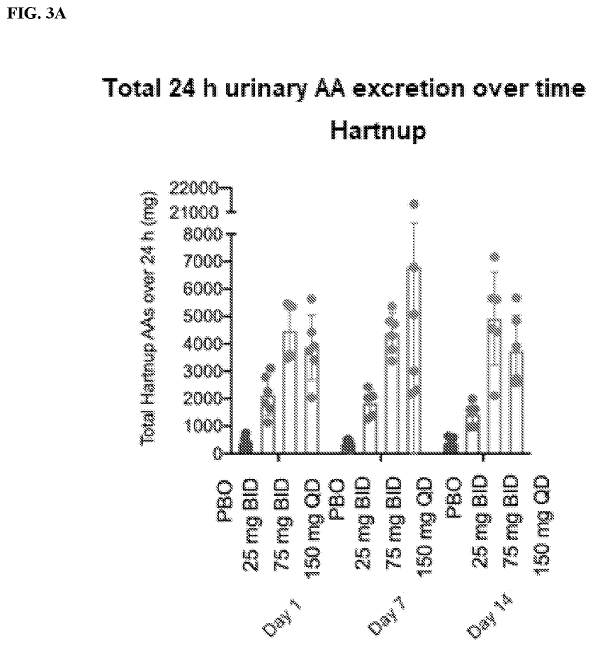

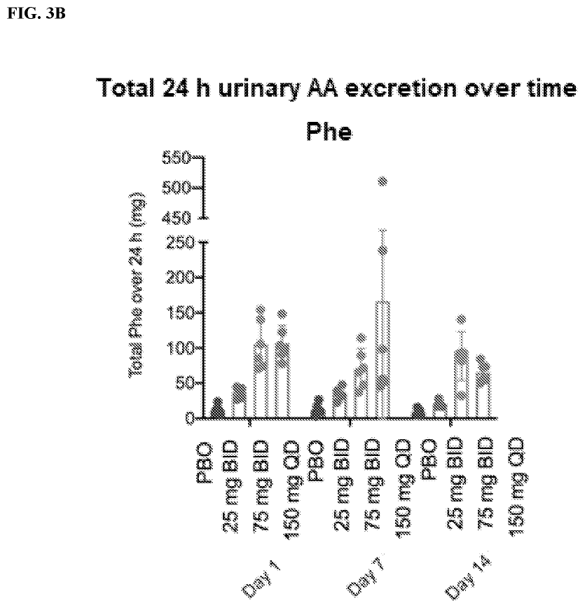

A suspension formulation of varying doses of Compound 1 (25 or 75 mg twice daily [BID] or 150 mg once daily [QD]) was administered to healthy volunteers. Changes in excretion of phenylalanine or overall SLC6A19 amino acid substrates (Hartnup amino acids) over 24 hours were measured at Day 1, 7 and 14 (FIGS. 3A and 3B). Urinary excretion of Hartnup amino acids over 24 hours increased from 0.4 g w/ PBO to 4.9 g w/ the compound 75 mg BID (13-fold increase) (Table 15A).

TABLE 15A
Hartnup Amino Acids

| Treatment (Dose) | Day | Mean | SD |
|---|---|---|---|
| Overall Placebo | Day 1 | 393.42 | 213.74 |
| | Day 7 | 360.30 | 144.55 |
| | Day 14 | 390.26 | 206.79 |
| Cpd 1 25 mg (BID) | Day 1 | 2123.44 | 738.29 |
| | Day 7 | 1835.21 | 494.26 |
| | Day 14 | 1427.77 | 448.67 |
| Cpd 1 75 mg (BID) | Day 1 | 4472.55 | 1010.31 |
| | Day 7 | 4398.31 | 723.71 |
| | Day 14 | 4922.75 | 1695.84 |
| Cpd 1 150 mg (QD) | Day 1 | 3861.43 | 1182.57 |
| | Day 7 | 6785.54 | 7348.84 |
| | Day 14 | 3737.47 | 1293.32 |

TABLE 15B
Phenylalanine

| Treatment (Dose) | Day | Mean | SD |
|---|---|---|---|
| Overall Placebo | Day 1 | 11.68 | 6.58 |
| | Day 7 | 12.49 | 8.14 |
| | Day 14 | 8.16 | 4.93 |
| Cpd 1 25 mg (BID) | Day 1 | 37.38 | 7.24 |
| | Day 7 | 35.22 | 9.10 |
| | Day 14 | 19.65 | 4.60 |
| Cpd 1 75 mg (BID) | Day 1 | 105.25 | 35.02 |
| | Day 7 | 70.98 | 27.99 |
| | Day 14 | 88.64 | 34.39 |
| Cpd 1 150 mg (QD) | Day 1 | 106.76 | 25.13 |
| | Day 7 | 166.71 | 183.48 |
| | Day 14 | 72.43 | 18.11 |

### Example 4. Comparison of Suspension and Tablet Formulations

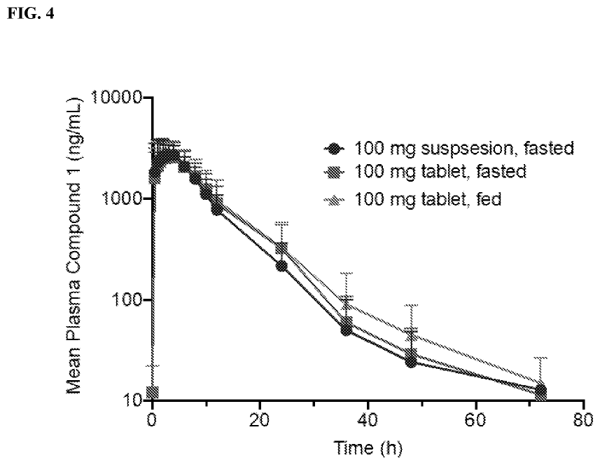

A suspension formulation (100 mg, fasted) and tablet formulation (100 mg, fasted or fed) of Compound 1 was administered to healthy volunteers. Plasma levels of Compound 1 over 72 was measured (FIG. 4). Post-dose plasma levels are recited in Table 16.

TABLE 16

| | | Mean | |
|---|---|---|---|
| Treatment (Dose) | Time Post-Dose (h) | (ng/mL) | SD |
| Cpd 1 100 mg suspsesion, fasted | 0 | | |
| | 0.5 | 1840.05 | 1703.13 |
| | 1 | 2308.62 | 1526.22 |
| | 1.5 | 2439.32 | 1412.75 |
| | 2 | 2630.41 | 1207.45 |
| | 2.5 | 2619.94 | 776.29 |
| | 3 | 2720.42 | 670.42 |
| | 4 | 2745.64 | 633.26 |
| | 6 | 2086.51 | 510.42 |
| | 8 | 1578.88 | 456.79 |
| | 10 | 1107.27 | 444.99 |
| | 12 | 778.14 | 303.69 |
| | 24 | 216.14 | 149.87 |
| | 36 | 50.00 | 50.68 |
| | 48 | 24.20 | 24.69 |
| | 72 | 12.85 | |
| Cpd 1 100 mg tablet, fasted | 0 | 12.11 | |
| | 0.5 | 1601.51 | 1276.73 |
| | 1 | 2109.71 | 1424.31 |
| | 1.5 | 2400.09 | 1460.81 |
| | 2 | 2545.58 | 1375.40 |
| | 2.5 | 2529.38 | 1227.07 |
| | 3 | 2575.85 | 1106.26 |
| | 4 | 2576.92 | 1023.01 |
| | 6 | 2068.78 | 857.61 |
| | 8 | 1652.52 | 584.01 |
| | 10 | 1252.07 | 513.93 |
| | 12 | 891.20 | 426.77 |
| | 24 | 323.51 | 235.49 |
| | 36 | 59.48 | 48.57 |
| | 48 | 29.02 | 22.91 |
| | 72 | 11.45 | |
| Cpd 1 100 mg tablet, fed | 0 | 12.14 | 9.99 |
| | 0.5 | 1656.19 | 1538.03 |
| | 1 | 2218.41 | 1447.50 |
| | 1.5 | 2317.32 | 1239.08 |
| | 2 | 2847.59 | 1103.25 |
| | 2.5 | 2909.96 | 721.63 |
| | 3 | 2953.49 | 714.14 |
| | 4 | 2855.13 | 871.47 |
| | 6 | 2259.44 | 789.48 |
| | 8 | 1737.60 | 731.01 |
| | 10 | 1270.61 | 622.47 |
| | 12 | 962.75 | 565.86 |
| | 24 | 328.73 | 255.39 |
| | 36 | 90.12 | 93.89 |
| | 48 | 44.96 | 43.58 |
| | 72 | 14.97 | 11.64 |

### INCORPORATION BY REFERENCE

All of the U.S. patents and U.S. and PCT published patent applications cited herein are hereby incorporated by reference.

### EQUIVALENTS

The foregoing written specification is sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method of treating or preventing a disease or disorder associated with a genetic defect in phenylalanine hydroxylase, comprising orally administering to a patient in need thereof an effective amount of the following compound, or a pharmaceutically acceptable salt thereof: 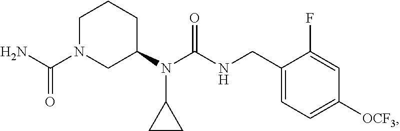 [SMILES: NC(=O)N1CCC[C@@H](N(C(=O)NCc2ccc(OC(F)(F)F)cc2F)C2CC2)C1]
wherein the compound is formulated as a tablet.

2. The method of claim 1, wherein the amount of compound is about 5 mg/day to about 1025 mg/day.

3. The method of claim 1, wherein the amount of compound is about 75 mg/day to about 1025 mg/day.

4. The method of claim 2, wherein the amount of the compound is about 100 mg/day to about 500 mg/day.

5. The method of claim 2, wherein the amount of the compound is about 500 mg/day to about 1000 mg/day.

6. The method of any one of claims 1-4, wherein the amount of the compound is about 100 mg/day to about 200 mg/day.

7. The method of any one of claims 1-4, wherein the amount of the compound is about 200 mg/day to about 300 mg/day.

8. The method of any one of claims 1-4, wherein the amount of the compound is about 300 mg/day to about 400 mg/day.

9. The method of any one of claims 1-4, wherein the amount of the compound is about 400 mg/day to about 500 mg/day.

10. The method of any one of claims 1-3 and 5, wherein the amount of the compound is about 500 mg/day to about 600 mg/day.

11. The method of any one of claims 1-3 and 5, wherein the amount of the compound is about 600 mg/day to about 700 mg/day.

12. The method of any one of claims 1-3 and 5, wherein the amount of the compound is about 700 mg/day to about 800 mg/day.

13. The method of any one of claims 1-3 and 5, wherein the amount of the compound is about 800 mg/day to about 900 mg/day.

14. The method of any one of claims 1-3 and 5, wherein the amount of the compound is about 900 mg/day to about 1000 mg/day.

15. The method of claim 4, wherein the amount of the compound is about 100 mg/day, about 125 mg/day, about 150 mg/day, or about 175 mg/day.

16. The method of claim 4, wherein the amount of the compound is about 200 mg/day, about 225 mg/day, about 250 mg/day, or about 275 mg/day.

17. The method of claim 4, wherein the amount of the compound is about 300 mg/day, about 325 mg/day, about 350 mg/day, or about 375 mg/day.

18. The method of claim 4, wherein the amount of the compound is about 400 mg/day, about 425 mg/day, about 450 mg/day, about 475 mg/day, or about 500 mg/day.

19. The method of claim 5, wherein the amount of the compound is about 500 mg/day, about 525 mg/day, about 550 mg/day, or about 575 mg/day.

20. The method of claim 5, wherein the amount of the compound is about 600 mg/day, about 625 mg/day, about 650 mg/day, or about 675 mg/day.

21. The method of claim 5, wherein the amount of the compound is about 700 mg/day, about 725 mg/day, about 750 mg/day, or about 775 mg/day.

22. The method of claim 5, wherein the amount of the compound is about 800 mg/day, about 825 mg/day, about 850 mg/day, or about 875 mg/day.

23. The method of claim 5, wherein the amount of the compound is about 900 mg/day, about 925 mg/day, about 950 mg/day, about 975 mg/day, or about 1000 mg/day.

24. The method of claim 1 or 2, wherein the amount of compound is about 5 mg/day to about 100 mg/day.

25. The method of any one of claims 1, 2, and 24, wherein the amount of the compound is about 5 mg/day to about 25 mg/day.

26. The method of any one of claims 1, 2, and 24, wherein the amount of the compound is about 25 mg/day to about 50 mg/day.

27. The method of any one of claims 1, 2, and 24, wherein the amount of the compound is about 50 mg/day to about 75 mg/day.

28. The method of any one of claims 1, 2, and 24, wherein the amount of the compound is about 5 mg/day, about 10 mg/day, about 25 mg/day, about 50 mg/day, or about 75 mg/day.

29. The method of any one of claims 1-28, wherein the % w/w of the compound in the tablet is about 5% to about 30% w/w.

30. The method of any one of claims 1-29, wherein the % w/w of the compound in the tablet is about 5% to about 10% w/w.

31. The method of any one of claims 1-29, wherein the % w/w of the compound in the tablet is about 20% to about 30% w/w.

32. The method of any one of claims 1-31, wherein each component of the tablet is intragranular.

33. The method of any one of claims 1-31, wherein at least one component of the tablet, other than the compound, is extragranular.

34. The method of any one of claims 1-33, wherein the tablet is a coated tablet.

35. The method of any one of claims 1-34, wherein the amount of the compound is administered in one dose per day.

36. The method of any one of claims 1-35, wherein the amount of the compound is administered in two equally divided doses per day.

37. The method of claim 24, wherein 25 mg of the compound is administered twice per day (BID).

38. The method of any one of claims 1-4, wherein 75 mg of the compound is administered twice per day (BID).

39. The method of any one of claims 1-4, wherein 150 mg of the compound is administered once per day (QD).

40. A method of treating or preventing a disease or disorder associated with a genetic defect in phenylalanine hydroxylase, comprising orally administering to a patient in need thereof an effective amount of the following compound, or a pharmaceutically acceptable salt thereof: 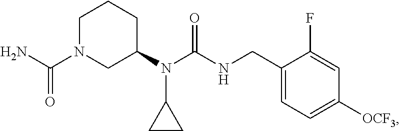 [SMILES: NC(=O)N1CCC[C@@H](N(C(=O)NCc2ccc(OC(F)(F)F)cc2F)C2CC2)C1]
wherein the compound is formulated as a suspension.

41. The method of claim 40, wherein the amount of compound is about 5 mg/day to about 1525 mg/day

42. The method of claim 40 or 41, wherein the amount of compound is about 10 mg/day to about 750 mg/day

43. The method of any one of claims 40-42, wherein the amount of the compound is about 10 mg/day to about 525 mg/day.

44. The method of any one of claims 40-43, wherein the amount of the compound is about 25 mg/day to about 100 mg/day.

45. The method of any one of claims 40-43, wherein the amount of the compound is about 150 mg/day to about 250 mg/day.

46. The method of any one of claims 40-43, wherein the amount of the compound is about 300 mg/day to about 500 mg/day.

47. The method of any one of claims 40-44, wherein the amount of the compound is about 25 mg/day.

48. The method of any one of claims 40-44, wherein the amount of the compound is about 75 mg/day.

49. The method of any one of claims 40-43 and 45, wherein the amount of the compound is about 150 mg/day.

50. The method of any one of claims 40-43 and 45, wherein the amount of the compound is about 250 mg/day.

51. The method of any one of claims 40-43 and 46, wherein the amount of the compound is about 300 mg/day.

52. The method of any one of claims 40-43 and 46, wherein the amount of the compound is about 500 mg/day.

53. The method of claim 40 or 41, wherein the amount of compound is about 750 mg/day to about 1000 mg/day

54. The method of any one of claims 40-43, wherein the amount of the compound is about 1000 mg/day to about 1500 mg/day.

55. The method of any one of claims 53-54, wherein the amount of the compound is about 1000 mg/day.

56. The method of any one of claims 53-54, wherein the amount of the compound is about 1500 mg/day.

57. The method of any one of claims 40-56, wherein the % w/w of the compound in the suspension is about 25% to about 95% w/w.

58. The method of any one of claims 40-57, wherein the % w/w of the compound in the t suspension is about 33% to about 60% w/w.

59. The method of any one of claims 40-57, wherein the % w/w of the compound in the suspension is about 75% to about 90% w/w.

60. The method of any one of claims 40-57, wherein the % w/w of the compound in the suspension is about 90% to about 95% w/w.

61. The method of any one of claims 40-60, wherein the suspension consists of the compound, a wetting agent, and a vehicle.

62. The method of any one of claims 40-61 wherein the amount of the compound is administered in one dose per day.

63. The method of any one of claims 40-61, wherein the amount of the compound is administered in two equally divided doses per day.

64. The method of any one of claims 40-44, wherein 25 mg of the compound is administered twice per day (BID).

65. The method of any one of claims 40-43, wherein 75 mg of the compound is administered twice per day (BID).

66. The method of any one of claims 40-43, wherein 150 mg of the compound is administered once per day (QD).

67. The method of any one of claims 1-66, wherein the treatment continues indefinitely.

68. The method of any one of claims 1-67, wherein the patient is a human.

69. The method of claim 68, wherein the patient is an adult.

70. The method of claim 62, wherein the patient is an elderly adult.

71. The method of claim 68, wherein the patient is a child.

72. The method of claim 68, wherein the patient is an infant or a toddler.

73. The method of any one of claims 68-72, wherein the patient is a male.

74. The method of any one of claims 68-72, wherein the patient is a female.

75. The method of any one of claims 68-74, wherein the human is of African ancestry, Asian ancestry, Caucasian ancestry, American Indian ancestry, Hispanic/Latino ancestry, or Pacific Islander ancestry.

76. The method of any one of claims 1-75, wherein the concentration of phenylalanine in the blood of the patient is reduced relative to pretreatment baseline.

77. The method of claim 76, wherein the concentration of phenylalanine is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

78. The method of claim 76, wherein the concentration of phenylalanine is reduced by at least 10%, at least 20%, at least 30%, at least 40%, at least 50%, at least 60%, at least 70%, at least 80%, or at least 90%.

79. The method of any one of claims 1-78, wherein the disease or disorder associated with a genetic defect in phenylalanine hydroxylase is phenylketonuria.

80. The method of claim 79, wherein the phenylketonuria is classical phenylketonuria.

81. The method of claim 79, wherein the phenylketonuria is mild phenylketonuria.

82. The method of any one of claims 1-78, wherein the disease or disorder associated with a genetic defect in phenylalanine hydroxylase is hyperphenylalaninemia.

83. The method of any one of claims 1-82, wherein the compound inhibits SLC6A19 in the patient.
